(19) United States
(12) Patent Application Publication
(10) Pub. No.: US 2010/0185100 A1
(43) Pub. Date: Jul. 22, 2010

(54) IDENTIFYING AND LOCALIZING TISSUE USING LIGHT ANALYSIS

(76) Inventors: Alexander Urban, Munich (DE); Alexander Druse, Munich (DE); Fritz Vollmer, Munich (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 12/689,282

(22) Filed: Jan. 19, 2010

Related U.S. Application Data
(60) Provisional application No. 61/147,825, filed on Jan. 28, 2009.

(30) Foreign Application Priority Data
Jan. 19, 2009 (EP) 09150848.1

(51) Int. Cl. A61B 6/00

(52) U.S. Cl. 600/475; 600/477

(57) ABSTRACT

The invention relates to a method for identifying and localizing body tissue, in which a light beam emitted from a tissue point or tissue region of interest is on the one hand positionally determined with the aid of an optical medical tracking system and is on the other hand analyzed with respect to its light properties, in order to determine from this the nature of the tissue point or tissue region. It also relates to a device for identifying and localizing body tissue, comprising: an optical medical tracking system which detects a light beam emitted from a tissue point or tissue region of interest and positionally determines the location of the tissue point or tissue region; and a light analyzer which analyses the light beam with respect to its light properties, in order to determine from this the nature of the tissue point or tissue region.

### RELATED APPLICATION DATA

This application claims the priority of U.S. Provisional Application No. 61/147,825, filed on Jan. 28, 2009, which is hereby incorporated in its entirety by reference.

### FIELD OF THE INVENTION

The present invention relates to identifying and localizing tissue for medical purposes. A method and a device are introduced for this purpose.

### BACKGROUND OF THE INVENTION

Identifying and localizing tissue and/or tissue parts, such as is the subject of the present invention, is highly important inter alia when detecting the delineations and/or boundaries and dimensions of tumors on a tissue in an area of interest. In accordance with the prior art such as is known for example from U.S. Pat. No. 5,699,798, an area of interest is illuminated using an illumination source which emits electromagnetic radiation at least one wavelength which interacts with a dye. The electromagnetic radiation has a wavelength of about 450 nm to about 2500 nm.

Another approach, in accordance with EP 1 795 142 B1, is to combine a medical tracking system with a video or infrared camera system and at least one localizable gamma camera in order to detect gamma radiation emitted by a tracer material.

The two cited methods in accordance with the prior art are costly (dyeing the tissue; gamma camera).

US 2006/0058683 A1 describes an optical examination system for biological tissue, without localization.

### SUMMARY OF THE INVENTION

It is the object of the present invention to optimize identifying and localizing tissue or tissue parts and/or tissue regions/points.

In particular, the intention is to provide information on the tissue positionally exactly and without too great a cost.

This object is solved in accordance with the invention by a method for identifying and localizing body tissue, in which a light beam emitted from a tissue point or tissue region of interest is on the one hand positionally determined with the aid of an optical medical tracking system and is on the other hand analyzed with respect to its light properties, in order to determine from this the nature of the tissue point or tissue region, and by a device for identifying and localizing body tissue, comprising: an optical medical tracking system which detects a light beam emitted from a tissue point or tissue region of interest and positionally determines the location of the tissue point or tissue region; and a light analyzer which analyses the light beam with respect to its light properties, in order to determine from this the nature of the tissue point or tissue region. The sub-claims define preferred embodiments of the invention.

A method for identifying and localizing body tissue is thus provided in accordance with the invention, in which a light beam emitted from a tissue point or tissue region of interest (for example by reflection of a light beam which hits the tissue or by stimulated light emission by the tissue or by a dye which is concentrated in the tissue, such as for example fluorescence or phosphorescence) is on the one hand positionally determined with the aid of an optical medical tracking system and is on the other hand analyzed with respect to its light properties, in order to determine from this the nature of the tissue point or tissue region. Device parts used for this purpose include the tracking system, in particular a light receiver, and a light analyzer for establishing the properties of the light coming from the tissue.

In accordance with the invention, the light emitted from the tissue or the tissue point is thus simultaneously tested as to its characteristics, and the location from which the light is emitted is positionally determined. In other words, the light reflection or stimulated light beam which leaves the tissue again is analyzed in accordance with the invention with device-based assistance, in order to be able to deduce the properties of the tissue. For this purpose, the tissue need not necessarily be treated in any special way (for example, dyed) beforehand, and costly devices such as gamma cameras are also not necessary. Either the type of ambient light which hits the tissue is known (for example, the illumination in the operating theatre) or defined light is radiated onto the region (for example, laser light or infrared light of an infrared tracking camera of the navigation system), so as to be able to deduce the tissue characteristics at individual, localizable regions from the reflection/emission. This enables boundaries and dimensions of regions of interest, for example tumor tissue, to be detected in a simply way. Tumor tissue can for example also be delineated using substances having defined emission/reflection properties, which are for example concentrated in tumor tissue due to their biological properties. Using such detection, it is then in turn possible to obtain or update information on areas to be treated, such that during the treatment, healthy tissue parts can be left alone and diseased tissue parts can be removed or treated, wherein the accuracy with which the location is detected, which is associated with a medical tracking system, directly benefits the patient. Pre-operative datasets can be updated and/or supplemented by the tissue information. In many operating theatres, medical tracking systems are already provided, such that the invention can be easily incorporated into existing hardware.

The light beam emitted from the tissue point or tissue region (the light beam can be a very narrow light beam or a planar light beam and/or a light beam having a greater cross-section) can be analyzed on the basis of a property such as its wavelength, luminosity, coherence, direction, scattering or a combination of such properties. Any property of the light which is altered by the tissue properties to be determined or by dyes which are introduced, and which can be verified using a corresponding analysis device, is in principle suitable.

In one embodiment of the present invention, a source light beam having defined, known light properties is radiated onto the tissue point or tissue region, and the difference between the properties of the source light beam and the light beam mentioned at the beginning (which is emitted and/or reflected by the tissue) is used as the basis for determining the nature of the tissue point or tissue region. The advantage of this is that it is possible to exactly know and/or set the properties of the source light beam, so as to also gather very high-quality information on the tissue from the reflected beam. The source light beam can be a laser light beam, in particular in the infrared, ultraviolet or visible light wavelength range. It can include light having known properties which hits the tissue over an area and/or a point light beam. The infrared light of the infrared tracking camera of the navigation system can also for example be used as the source light beam. When using point light beams, the position can be determined from the tracking system by means of simple triangulation. When using planar light emitters, structures which illuminate over an area are obtained, the spatial position of which can be detected via specialized stereoscopic video evaluations, wherein two stereoscopically arranged cameras can be used.

In accordance with one variant of the method in accordance with the invention, the light beam is analyzed on the basis of light-altering features of the tissue point or tissue region. The boundaries and/or dimensions of defined tissue portions can be detected positionally and with respect to the tissue properties, and the captured data can be provided to a medical navigation system, wherein it is in particular used to update the pre-operative image data.

The device in accordance with the invention for identifying and localizing body tissue includes an optical medical tracking system which detects an individual light beam or light beams which are emitted over an area, emitted from a tissue point or tissue region of interest. The tracking system positionally determines the location of the tissue point or tissue region, and the device also comprises a light analyzer which to analyses the light beam with respect to its light properties, in order to determine from this the nature of the tissue point or tissue region.

The device can include a source light emitter, in particular a laser light emitter as mentioned above, either as a point light emitter and/or a planar light emitter.

In one device embodiment in accordance with the invention, the part which receives the light beam, in particular a light sensor, specifically a laser light sensor, is arranged on the source light emitter, which in turn is positionally determined and/or tracked by the tracking system.

The data on the properties of the light beam and the source light beam, i.e. the nature of the tissue and the position of the tissue point, can be processed in a data processing unit which is in particular a part of a medical navigation system.

### BRIEF DESCRIPTION OF THE DRAWINGS

The invention is illustrated below in more detail, on the basis of example embodiments and by referring to the enclosed drawings. It can include any of the features described here, individually and in any expedient combination and can also in particular be embodied as a program which, when it is running on a computer or is loaded on a computer, causes the computer to perform a method such as has been described above in various embodiments. The invention also includes a computer program storage medium which comprises such a program.

FIG. 1 shows a schematic representation of a device setup which is used when implementing the present invention.

FIG. 2 shows a tissue region of interest, with light irradiation/reflection in accordance with the invention.

### DETAILED DESCRIPTION

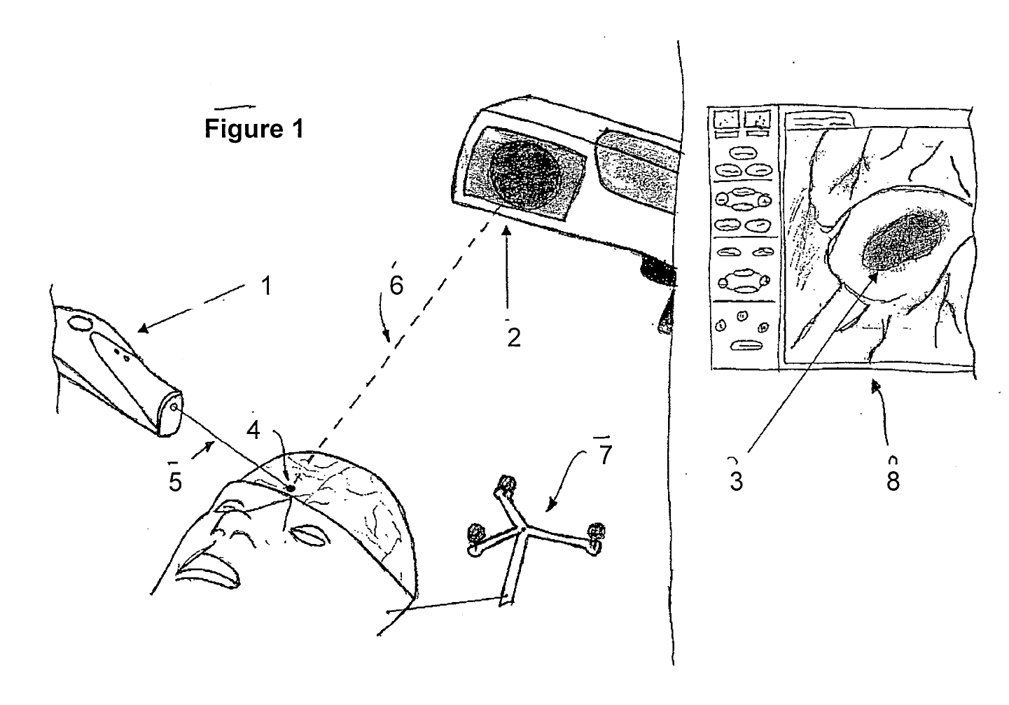

The device parts shown in FIG. 1 are: a laser point emitter 1; a medical tracking system 2 comprising a camera; and a reference array 7 which is connected to a patient's head and the position of which can thus be detected with the aid of the tracking system 2. The tracking system 2 can also detect a light point (reflection point 4), the light beam of which towards the camera lens has been provided with the reference sign 6. Because the tracking system 2 comprises two cameras and can therefore detect two light beams 6, the position of the light point 4 can be three-dimensionally detected, and since the position of the patient is detected via the reference array 7, it is also then known where the point 4 lies on the patient.

The light point 4 is created by emitting a light beam 5 by means of the integrated laser emitter 1 which in the present case is a hand-held laser emitter.

The screen output 8 of a navigation system is also shown in FIG. 1, separated off on the right-hand side, wherein the reference sign 8 can also designate the navigation system together with the screen output. The image 3 is an image which has been generated with the aid of the information from the method in accordance with the invention and is provided to the surgeon as an aid.

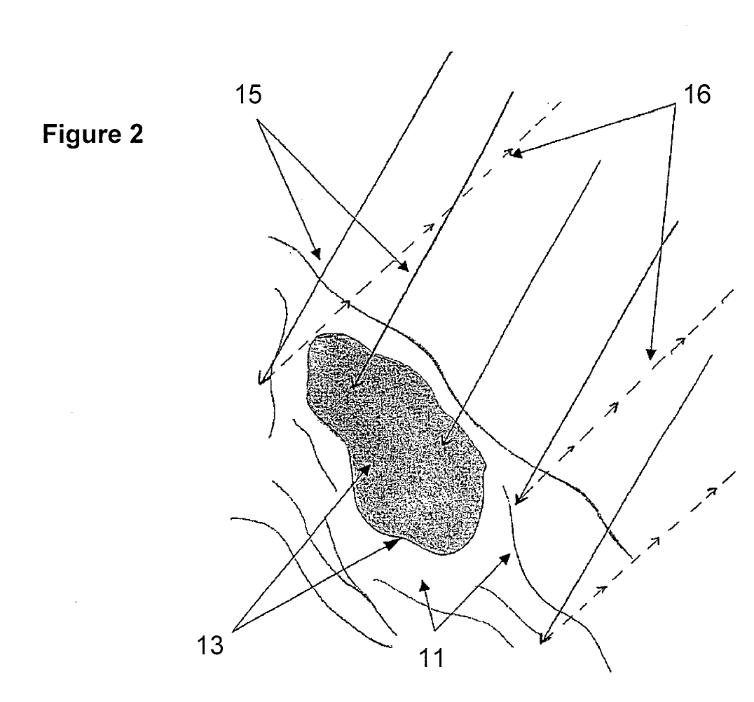

In FIG. 2, the reference sign 11 designates a tissue region of interest, the reference sign 13 indicates a tumor portion and its boundary, and the reference sign 15 designates incident light beams (source light) while the reference sign 16 indicates reflected light which can be “read off” in accordance with the invention.

The integrated laser 1 shown in FIG. 1 can emit one or more laser beams 5 at a defined wavelength. The reflected laser beam 6 from the point 4 is analyzed, and is firstly captured by the cameras of the tracking system 2 for this purpose. The beam 6 is analyzed using a software of the navigation system, i.e. the software identifies the properties and composition of the reflected/emitted light which is emitted from the tissue, from which it is possible to identify the type of tissue which is present at the point 4. This enables data material captured beforehand to be updated (during the operation), even repeatedly in succession, as often as is necessary.

It is possible to emit laser light which covers a number of wavelengths (for example, the laser light 15 in FIG. 2), in order to test how different wavelengths are reflected (the reflection 16 in FIG. 2). The positions of the tumor tissue 13 and its delineation with respect to the surrounding healthy tissue 11 can then be identified from this information. This of course works on the one hand using a source light beam which is applied over an area or a source light beam having a greater diameter, or also on the other hand using a point light beam (FIG. 1) which is moved over the tissue, and the reflections of which are analyzed sequentially.

As already noted above, the laser can operate in the infrared range, the ultraviolet range or the visible light range. One interesting embodiment of a device in accordance with the invention includes a rigid fiber optic which is arranged on a handle, such as has for example been designated in FIG. 1 by the reference sign 1. In this case, however, said handle would be provided with a tracking reference for the tracking system 2, thus allowing the fiber ends to be navigated and/or tracked. These fibers and/or fiber ends are used to guide the source light for illuminating the tissue which is being examined, while on the other hand, these or also other fibers in the to front part of the handle also receive the reflected light and relay it to the detector/analysis device. Thus, even weak signals or alterations to the light by the tissue to be examined can be evaluated.

Using the method in accordance with the invention and/or the device in accordance with the invention, it is possible to detect not only tumors and their boundaries but also other surface structures such as for example the surface of bones or birthmarks.

Computer program elements of the invention may be embodied in hardware and/or software (including firmware, resident software, micro-code, etc.). The computer program elements of the invention may take the form of a computer program product which may be embodied by a computer-usable or computer-readable storage medium comprising computer-usable or computer-readable program instructions, “code” or a “computer program” embodied in said medium for use by or in connection with the instruction executing system. Within the context of this application, a computer-usable or computer-readable medium may be any medium which can contain, store, communicate, propagate or transport the program for use by or in connection with the instruction executing system, apparatus or device. The computer-usable or computer-readable medium may for example be, but is not limited to, an electronic, magnetic, optical, electromagnetic, infrared or semiconductor system, apparatus, device or medium of propagation, such as for example the Internet. The computer-usable or computer-readable medium could even for example be paper or another suitable medium on which the program is printed, since the program could be electronically captured, for example by optically scanning the paper or other suitable medium, and then compiled, interpreted or otherwise processed in a suitable manner. The computer program product and any software and/or hardware described here form the various means for performing the functions of the invention in the example embodiment(s).

the invention has been shown and described with respect to one or more particular preferred embodiments, it is clear that equivalent amendments or modifications will occur to the person skilled in the art when reading and interpreting the text and enclosed drawing(s) of this specification. In particular with regard to the various functions performed by the elements (components, assemblies, devices, compositions, etc.) described above, the terms used to describe such elements (including any reference to a “means”) are intended, unless expressly indicated otherwise, to correspond to any element which performs the specified function of the to element described, i.e. which is functionally equivalent to it, even if it is not structurally equivalent to the disclosed structure which performs the function in the example embodiment(s) illustrated here. Moreover, while a particular feature of the invention may have been described above with respect to only one or some of the embodiments illustrated, such a feature may also be combined with one or more other features of the other embodiments, in any way such as may be desirable or advantageous for any given application of the invention.

What is claimed is:

1. A method for identifying and localizing body tissue, in which a light beam emitted from a tissue point or tissue region of interest is on the one hand positionally determined with the aid of an optical medical tracking system and is on the other hand analyzed with respect to its light properties, in order to determine from this the nature of the tissue point or tissue region.

2. The method according to claim 1, wherein the light beam is analyzed on the basis of a property such as its:
wavelength;
luminosity;
coherence;
direction;
scattering;
or a combination of such properties.

3. The method according to claim 1, wherein a source light beam having defined, known light properties is radiated onto the tissue point or tissue region, and the difference between the properties of the source light beam and the light beam is used as the basis for determining the nature of the tissue point or tissue region.

4. The method according to claim 3, wherein the source light beam is a laser light beam.

5. The method according to claim 4, wherein the laser light beam is in the infrared, ultraviolet or visible light wavelength range.

6. The method according to claim 3, wherein the source light beam is a light having known properties which hits the tissue over an area.

7. The method according to claim 3, wherein the source light beam is a point light beam.

8. The method according to claim 1, wherein the light beam is analyzed on the basis of light-altering features of the tissue point or tissue region.

9. The method according to claim 8, wherein the light-altering features are bodily features.

10. The method according to claim 1, wherein boundaries and/or dimensions of defined tissue portions are detected positionally and with respect to the tissue properties, and the captured data is provided to a medical navigation system.

11. The method according to claim 10, wherein the captured data is used to update data material captured beforehand.

12. A device for identifying and localizing body tissue, comprising: an optical medical tracking system which detects a light beam emitted from a tissue point or tissue region of interest and positionally determines the location of the tissue point or tissue region; and a light analyzer which analyses the light beam with respect to its light properties, in order to determine from this the nature of the tissue point or tissue region.

13. The device according to claim 12, wherein it includes a source light emitter.

14. The device according to claim 13, wherein the source light emitter is a laser light emitter.

15. The device according to claim 14, wherein the laser light emitter radiates in the infrared, ultraviolet or visible light wavelength range.

16. The device according to claim 13, wherein the source light emitter is a point light emitter.

17. The device according to claim 13, wherein the source light emitter is a planar light emitter.

18. The device according to claim 13, wherein the part which receives the light beam is arranged on the source light emitter, which in turn is positionally determined and/or tracked by the tracking system.

19. The device according to claim 18, wherein the part which receives the light beam is a light sensor.

20. The device according to claim 19, wherein the light sensor is a laser light sensor.

21. The device according to claim 13, wherein the data on the properties of the light beam and the source light beam is processed in a data processing unit.

22. The device according to claim 21, wherein the data processing unit is a part of a medical navigation system.

23. A program which, when it is running on a computer or is loaded on a computer, causes the computer to perform a method in accordance with claim 1.

24. A computer program storage medium which comprises a program according to claim 23.
